(19) United States
(12) Patent Application Publication
(10) Pub. No.: US 2025/0177457 A1
(43) Pub. Date: Jun. 5, 2025

(54) SUPPLEMENT COMPOSITION FOR CONSUMPTION BY INDIVIDUALS BEING ADMINISTERED A GLP-1 AGONIST

(71) Applicant: Food Sciences Corp., Mount Laurel, NJ (US)

(72) Inventors: Rosemary WHITE, Haddonfield, NJ (US); Laura DAEHNKE, Philadelphia, PA (US); Lauren SITEMAN, Galloway, NJ (US); Clare GAMBINO, Glassboro, NJ (US)

(21) Appl. No.: 18/527,538

(22) Filed: Dec. 4, 2023

(51) Int. Cl. A61K 35/742; A61K 9/00; A61K 31/07; A61K 31/122; A61K 31/198; A61K 31/22; A61K 31/352; A61K 31/375; A61K 31/4188; A61K 31/4415; A61K 31/455; A61K 31/51; A61K 31/519; A61K 31/525; A61K 31/593; A61K 31/714; A61K 33/06; A61K 33/18; A61K 33/24; A61K 33/26; A61K 33/30; A61K 33/32; A61K 33/42; A61K 35/20; A61K 36/484; A61K 36/55; A61K 38/46; A61K 38/47; A61K 38/48; A61K 47/26; A61K 47/36; A61K 47/42; A61K 47/46

(52) U.S. Cl. CPC A61K 35/742; A61K 9/0095; A61K 31/07; A61K 31/122; A61K 31/198; A61K 31/22; A61K 31/352; A61K 31/375; A61K 31/4188; A61K 31/4415; A61K 31/455; A61K 31/51; A61K 31/519; A61K 31/525; A61K 31/593; A61K 31/714; A61K 33/06; A61K 33/18; A61K 33/24; A61K 33/26; A61K 33/30; A61K 33/32; A61K 33/42; A61K 35/20; A61K 36/484; A61K 36/55; A61K 38/465; A61K 38/47; A61K 38/48; A61K 47/26; A61K 47/36; A61K 47/42; A61K 47/46

(57) ABSTRACT

A composition for consumption by an individual who is being treated with a GLP-1 agonist, including: at least one amino acid source in an amount effective to alleviate loss of lean body mass as a side effect of being treated with the GLP-1 agonist; at least one dietary fiber; and a therapeutically effective amount of at least one of a bioactive anti-emetic agent, an anti-bloating agent, an anti-diarrheal agent and an anti-constipation agent, wherein the composition is a premix or a ready-to-drink supplement effective to alleviate at least one additional side effect of being treated with the GLP-1 agonist, which is selected from nausea, bloating, vomiting, diarrhea and constipation. A method for alleviating loss of lean body mass and at least one additional side effect of being administered a GLP-1 agonist, includes feeding the composition to an individual who is being administered the GLP-1 agonist.

### BACKGROUND OF THE INVENTION

### 1. Field of Invention

This invention relates to supplements and to the dietary needs of individuals undergoing GLP-1 agonist therapy.

### 2. Description of Related Art

GLP-1 receptor agonists (sometimes referred to herein as “GLP-1 agonists”) are a class of medications used to treat type 2 diabetes by mimicking the action of a natural hormone called glucagon-like peptide-1 (GLP-1). These drugs stimulate the release of insulin, lower blood sugar levels, and help regulate appetite, making them effective for both glycemic control and weight management, particularly but not exclusively in diabetic patients.

GLP-1 agonist medications currently available in the U.S. include Dulaglutide (TRULICITY), Exenatide (BYETTA), Liraglutide (VICTOZA and SAXENDA), Lixisenatide (ADLYXIN) and Semaglutide (WEGOVY, OZEMPIC, and RYBELSUS).

While these medications have proven to be effective for blood glucose control and weight loss, they also affect digestion and food choices that can significantly alter diet quality and compromise nutritional status. The impact of reduced diet quality can have short-term and long-term consequences on metabolism and overall health for weight loss patients. Studies on these medications highlight issues that negatively impact nutritional health and wellbeing, including:
Loss of Lean Body Mass, accounting for 40% or more of total weight loss;
Gastrointestinal side effects including nausea, vomiting, bloating, diarrhea, and constipation;
Altered food preferences which reduce the intake of protein, fiber, and essential micronutrients;
Significant decreases in daily calorie intake by as much as 40-50%; and
Reduced micronutrient status and altered gut microbiome due to slowed gastric emptying and digestive changes.

U.S. Patent Application Publication No. 20220218768 A1 discloses a method of treating type 1 diabetes or type 2 diabetes or one or more complications thereof in a subject in need thereof comprising administering to the subject a therapeutically effective combination of at least one Lactobacillus probiotic strain and an incretin-based drug, such as a GLP-1 receptor agonist, wherein administration of the combination results in enhanced therapeutic efficacy relative to the administration of the incretin-based drug alone.

Despite the foregoing development, there remains a need for improving treatment with GLP-1 receptor agonists, particularly through the reduction of unwanted side-effects.

All references cited herein are incorporated herein by reference in their entireties.

### BRIEF SUMMARY OF THE INVENTION

Accordingly, a first aspect of the invention is a composition for consumption by an individual who is being treated with a GLP-1 agonist, said composition comprising: at least one amino acid source in an amount effective to alleviate loss of lean body mass as a side effect of being treated with the GLP-1 agonist; at least one dietary fiber; and a therapeutically effective amount of at least one member selected from the group consisting of a bioactive anti-emetic agent, an anti-bloating agent, an anti-diarrheal agent and an anti-constipation agent, wherein the composition is a premix or a ready-to-drink supplement effective to alleviate at least one additional side effect of being treated with the GLP-1 agonist, said at least one additional side effect being selected from the group consisting of nausea, bloating, vomiting, diarrhea and constipation.

In certain embodiments, the composition further comprises vitamins, minerals and at least one probiotic.

In certain embodiments, the composition further comprises at least one alpha-linoleic acid (ALA) source, at least one medium chain triglyceride (MCT) having a chain length of 6-12 carbons, leucine, Glycyrrhiza glabra extract, at least one zinc compound, amylase, protease, lactase, cellulase and lipase.

In certain embodiments, the composition comprises: at least one potassium compound; at least one magnesium compound; at least one choline compound; at least one vitamin; at least one iron compound; at least one probiotic; at least one manganese compound; at least one copper compound; at least one chromium compound; at least one molybdenum compound; at least one iodine compound; and at least one selenium compound, wherein the composition is safe for human consumption.

In certain embodiments, the at least one amino acid source comprises 12-28 grams per 6 oz (177.4 ml) serving of at least one protein, and the at least one dietary fiber comprises at least one prebiotic.

In certain embodiments, the at least one amino acid source comprises whey protein concentrate and whey protein isolate; the at least one calcium compound is milk calcium complex; the at least one dietary fiber is a soluble fiber that is a digestion-resistant maltodextrin prebiotic; the at least one potassium compound is dipotassium phosphate; the at least one magnesium compound comprises magnesium bisglycinate dihydrate and magnesium lactate; the at least one choline compound is choline bitartrate; the at least one vitamin comprises ascorbic acid, alpha tocopherol acetate, niacinamide, pyridoxine hydrochloride, vitamin A palmitate, riboflavin, thiamin mononitrate, folic acid, phytonadione, cholecalciferol, menaquinone 7 and cyanocobalamin; the at least one iron compound is ferrous sulfate; the at least one probiotic is Bacillus Coagulans MTCC 5856; the at least one manganese compound is manganese sulfate; the at least one copper compound is copper gluconate; the at least one chromium compound is chromium polynicotinate; the at least one molybdenum compound is molybdenum glycinate chelate; the at least one iodine compound is potassium iodide; and the at least one selenium compound is L-Selenomethionine.

In certain embodiments, the composition further comprises at least one alpha-linoleic acid (ALA) source, at least one medium chain triglyceride (MCT) having a chain length of 6-12 carbons, leucine, Glycyrrhiza glabra extract, at least one zinc compound, amylase, protease, lactase, cellulase and lipase.

In certain embodiments, the at least one ALA source is flaxseed; the at least one MCT comprises at least one member selected from the group consisting of caproic acid, caprylic acid, capric acid and lauric acid; and the at least one zinc compound comprises zinc carnosine and zinc sulfate.

In certain embodiments, each serving of the composition comprises:

| Ingredient | Min (g) | Max (g) |
|---|---|---|
| Whey Protein Concentrate | 5.000000 | 20.000000 |
| Whey Protein Isolate | 5.000000 | 20.000000 |
| Digestion-Resistant Maltodextrin | 1.000000 | 8.000000 |
| Flaxseed | >0.000000 | 5.000000 |
| Dipotassium Phosphate | 0.960000 | 2.980000 |
| Milk Calcium Complex | 1.250000 | 2.500000 |
| Magnesium Bisglycinate Dihydrate | 0.375000 | 1.500000 |
| Magnesium Lactate | 0.375000 | 1.500000 |
| Choline Bitartrate | 0.370000 | 1.480000 |
| Leucine | >0.000000 | 1.000000 |
| Medium Chain Triglycerides | >0.000000 | 0.730000 |
| Ascorbic Acid | 0.277941 | 0.555882 |
| Glycyrrhiza Glabra Extract | >0.000000 | 0.075000 |
| Ferrous Sulfate | 0.030484 | 0.060968 |
| Zinc Carnosine | >0.000000 | 0.025000 |
| Alpha Tocopherol Acetate | 0.009499 | 0.018998 |
| Niacinamide | 0.004703 | 0.009406 |
| Amylase | >0.000000 | 0.008850 |
| Bacillus Coagulans MTCC 5856 | 0.002150 | 0.008600 |
| Manganese Sulfate | 0.001896 | 0.007582 |
| Copper Gluconate | 0.001890 | 0.007560 |
| D-Calcium Pantothenate | 0.001595 | 0.006378 |
| Protease | >0.000000 | 0.004650 |
| Lactase | >0.000000 | 0.003375 |
| Pyridoxine Hydrochloride | 0.000592 | 0.002368 |
| Vitamin A Palmitate | 0.000555 | 0.002220 |
| Cellulase | >0.000000 | 0.002168 |
| Riboflavin | 0.000395 | 0.001580 |
| Thiamin Mononitrate | 0.000368 | 0.001472 |
| Lipase | >0.000000 | 0.001200 |
| Zinc Sulfate | >0.000000 | 0.001116 |
| Chromium Polynicotinate | 0.000092 | 0.000368 |
| Molybdenum Glycinate Chelate | 0.000083 | 0.000330 |
| Folic Acid | 0.000080 | 0.000320 |
| Potassium Iodide | 0.000059 | 0.000236 |
| L-Selenomethionine | 0.000036 | 0.000144 |
| Phytonadione | 0.000035 | 0.000138 |
| Cholecalciferol | 0.000012 | 0.000048 |
| Biotin | 0.000009 | 0.000034 |
| Magnesium Carbonate | 0.000065 | 0.000026 |
| Menaquinone 7 | 0.000065 | 0.000026 |
| Cyanocobalamin | 0.000002 | 0.000006 |

In certain embodiments, the composition is a readily soluble powder premix effective to combine with water to provide a drinkable emulsion.

In certain embodiments, the composition is a ready-to-drink emulsion further comprising water.

In certain embodiments, the composition is effective to alleviate loss of lean body mass, nausea, bloating, vomiting, diarrhea and constipation.

In certain embodiments, the composition comprises each of the bioactive anti-emetic agent, the anti-bloating agent, the anti-diarrheal agent and the anti-constipation agent, wherein: the bioactive anti-emetic agent comprises at least one member selected from the group consisting of at least one digestive enzyme, Glycyrrhiza glabra and zinc carnosine; the anti-bloating agent comprises at least one member selected from the group consisting of Glycyrrhiza glabra, at least one probiotic and at least one digestive enzyme; the anti-diarrheal agent is at least one member selected from the group consisting of at least one dietary fiber and at least one probiotic; and the anti-constipation agent is at least one member selected from the group consisting of at least one dietary fiber and at least one probiotic.

A second aspect of the invention is a method for alleviating loss of lean body mass and at least one additional side effect of being administered a GLP-1 agonist, said method comprising feeding the composition of the invention to an individual who is being administered the GLP-1 agonist.

In certain embodiments of the method, the at least one additional side effect is a member selected from the group consisting of nausea, bloating, vomiting, diarrhea and constipation.

In certain embodiments, the method alleviates loss of lean body mass, nausea, bloating, vomiting, diarrhea and constipation.

In certain embodiments of the method, the composition is fed to the individual in two separate daily servings of a liquid emulsion each of which has a volume of less than 8 ounces (236.6 ml).

In certain embodiments of the method, the at least one amino acid source of the composition comprises 12-28 grams per 6 oz (177.4 ml) serving of at least one protein, and the at least one dietary fiber comprises at least one prebiotic.

In certain embodiments of the method, each serving comprises:

| Ingredient | Min (g) | Max (g) |
|---|---|---|
| Whey Protein Concentrate | 5.000000 | 20.000000 |
| Whey Protein Isolate | 5.000000 | 20.000000 |
| Digestion-Resistant Maltodextrin | 1.000000 | 8.000000 |
| Flaxseed | >0.000000 | 5.000000 |
| Dipotassium Phosphate | 0.960000 | 2.980000 |
| Milk Calcium Complex | 1.250000 | 2.500000 |
| Magnesium Bisglycinate Dihydrate | 0.375000 | 1.500000 |
| Magnesium Lactate | 0.375000 | 1.500000 |
| Choline Bitartrate | 0.370000 | 1.480000 |
| Leucine | >0.000000 | 1.000000 |
| Medium Chain Triglycerides | >0.000000 | 0.730000 |
| Ascorbic Acid | 0.277941 | 0.555882 |
| Glycyrrhiza Glabra Extract | >0.000000 | 0.075000 |
| Ferrous Sulfate | 0.030484 | 0.060968 |
| Zinc Carnosine | >0.000000 | 0.025000 |
| Alpha Tocopherol Acetate | 0.009499 | 0.018998 |
| Niacinamide | 0.004703 | 0.009406 |
| Amylase | >0.000000 | 0.008850 |
| Bacillus Coagulans MTCC 5856 | 0.002150 | 0.008600 |
| Manganese Sulfate | 0.001896 | 0.007582 |
| Copper Gluconate | 0.001890 | 0.007560 |
| D-Calcium Pantothenate | 0.001595 | 0.006378 |
| Protease | >0.000000 | 0.004650 |
| Lactase | >0.000000 | 0.003375 |
| Pyridoxine Hydrochloride | 0.000592 | 0.002368 |
| Vitamin A Palmitate | 0.000555 | 0.002220 |
| Cellulase | >0.000000 | 0.002168 |
| Riboflavin | 0.000395 | 0.001580 |
| Thiamin Mononitrate | 0.000368 | 0.001472 |
| Lipase | >0.000000 | 0.001200 |
| Zinc Sulfate | >0.000000 | 0.001116 |
| Chromium Polynicotinate | 0.000092 | 0.000368 |
| Molybdenum Glycinate Chelate | 0.000083 | 0.000330 |
| Folic Acid | 0.000080 | 0.000320 |
| Potassium Iodide | 0.000059 | 0.000236 |
| L-Selenomethionine | 0.000036 | 0.000144 |
| Phytonadione | 0.000035 | 0.000138 |
| Cholecalciferol | 0.000012 | 0.000048 |
| Biotin | 0.000009 | 0.000034 |
| Magnesium Carbonate | 0.000065 | 0.000026 |
| Menaquinone 7 | 0.000065 | 0.000026 |
| Cyanocobalamin | 0.000002 | 0.000006 |

### DETAILED DESCRIPTION OF PREFERRED EMBODIMENTS OF THE INVENTION

### Glossary

Throughout the description, where compositions are described as: having, including or comprising specific components or where processes are described as: having, including or comprising specific process steps, it is contemplated that compositions of the present teachings also consist essentially of, or consist of, the recited components and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

Except when noted, the terms “individual”, “subject” and “patient” are used interchangeably and refer to mammals such as human patients and non-human primates, as well as experimental animals such as: dogs, rabbits, rats, mice and other animals. Accordingly, the terms “individual”, “subject” and “patient” as used herein refer to a living being to which the composition of the invention can be administered.

It is to be understood that when referring to the inventive composition, “supplement,” “nutritional supplement,” “dietary supplement,” and “composition” all refer to a composition for consumption by an individual who is being treated with a GLP-1 agonist. It is also to be understood that the aforementioned terms, unless specified otherwise, refer to the composition in any physical form including a powder, gel, solid, emulsion, or a liquid.

As used herein, “beverage” refers to beverages of water-like consistency as well as emulsions unless specified otherwise.

As used herein, “suitable for beverage use”, allowing for “easier use in beverage formulations”, and like phrases refer to preparing the composition for stable incorporation into ready-made liquids, emulsions, or powders that may be later added to a liquid, without diminishing the effect of the active ingredients within the composition.

As used herein, “sources of protein” refers to any suitable protein utilized in nutritional formulations.

As used herein, “probiotics” are understood the refer to live microorganisms which promote health benefits when consumed by a human being, generally by improving or restoring the gut microbiota.

As used herein, “dietary fiber” refers to the portion of plant-derived food that cannot be completely broken down by human digestive enzymes.

As used herein, “digestive enzymes” refers to proteins suitable for use in nutritional formulations that break down food and aid in proper digestion via breaking down polymeric macromolecules into their smaller building blocks.

“Readily digestible” as used herein is used to describe a nutritional substance which quickly absorbs into the body within approximately 1-2 hours.

Finally, as used herein, “primary ingredients” refers to nutritive ingredients that make up the majority percentage of the composition, “secondary ingredients” refers to non-nutritive ingredients making up a lesser total percentage of the composition than the primary ingredients, and “tertiary additives” refers to indirect additives making up the lowest total percentage of the composition.

The invention is drawn to a composition in the form of a supplement for users of GLP-1 agonist drugs, particularly GLP-1 diabetes and anti-obesity medications. As users of GLP-1 agonist drugs frequently experience nutritional deficiencies and gastrointestinal symptoms as side effects of the drugs, the disclosed supplement includes ingredients such as proteins, vitamins and minerals, flaxseed, leucine, digestive enzymes, and readily digestible probiotics to remedy dietary deficiencies while also encouraging efficient digestion.

Including an enriched protein supplement when utilizing GLP-1 anti-obesity medications ensures essential dietary nutrients are available. The effects of slower gastric emptying are offset by utilizing more absorbable nutritional ingredients. Consuming readily available nutrients is crucial in sustaining biochemical activities of all systems in the body during weight loss. In certain embodiments, the composition is in a form selected from the group including a powder, gel, solid, emulsion, and a liquid. In certain exemplary embodiments, the gel is in the form of desserts, confections, sauces, edible gelatin, or similar gel substances. In other exemplary embodiments, the solid is in the form of baked goods or nutritional bars. In further exemplary embodiments, the emulsion is in the form of a shake, smoothie, or similar viscous substance, and the liquid is in the form of a liquid beverage sold in ready-to-drink form. In preferred embodiments, the composition is in the form of a powder, e.g., a premix that is readily soluble and may later be mixed with a liquid, such as water, to obtain a beverage. The composition includes the following ingredients selected with specific focus on the digestive and nutritional impact of anti-obesity medications.

By providing a select combination of vitamins, minerals, protein, healthy fats, digestive enzymes, dietary fiber, and probiotics, the composition of the invention is a supplement that in certain examples provides 50% or more of 25 essential micronutrients in one serving. As a result, patients on GLP-1 anti-obesity medications are able to fully supplement their required nutrients in as little as two servings or less of the supplement.

In preferred embodiments, the composition includes at least one of a bioactive anti-emetic agent, an anti-bloating agent, an anti-diarrheal agent and an anti-constipation agent. Moreover, the composition preferably alleviates at least one side effect common to taking GLP-1 agonists, including nausea, bloating, vomiting, diarrhea and constipation. Anti-emetic agents, e.g., substances that reduce feelings of nausea, are provided in certain embodiments of the composition in the form of at least one of digestive enzymes, Glycyrrhiza glabra extract, and zinc carnosine. In certain embodiments, anti-bloating agents are provided in the composition in the form of at least one of digestive enzymes, Glycyrrhiza glabra extract, and a probiotic. In further embodiments, anti-diarrheal agents are provided in the composition in the form of at least one of a probiotic and dietary fiber. In still further embodiments, anti-constipation agents are provided in the composition in the form of at least one of a probiotic and dietary fiber.

### Whey Protein Concentrate and Whey Protein Isolate Blend

The supplement is protein-enriched for improved nutrient absorption and gut health. The protein is preferably a high-quality dairy protein selected from the group including individual amino acids and a protein blend that that would meet a high protein quality score, such as a protein digestibility-corrected amino acid score (PDCAAS) equal to 1.0. In certain embodiments, the high-quality dairy protein is selected from the group including but not limited to whey protein isolate, whey protein concentrate, caseinates including sodium, calcium and potassium forms, egg protein including albumin, plant proteins including soy, pea, and hydrolyzed plant protein, and individual amino acids to meet the amino acid balance for human protein synthesis. In preferred embodiments, the proteins include easily digested dairy proteins to provide the essential amino acids at the proportions necessary to support protein synthesis and to preform functionally and palatably to achieve a serving size of less than eight fluid ounces when in reconstituted form. In most preferred embodiments, the proteins include highly absorbable whey proteins in a serving size of less than six fluid ounces. High-quality dairy protein such as whey protein offsets the significant dietary deficit associated with reduced calorie and delayed intestinal access. Whey proteins are rapidly digested compared to other proteins, ensuring the dietary supply of amino acid building blocks to protect lean body mass and sustained energy levels. GLP-1 reduces the rate of digestion, and whey proteins are well-suited to support the physiological demand to maintain muscle integrity and essential metabolic functions.

Use of a selected blend of whey proteins with added leucine optimizes amino acid bioavailability for lean muscle (e.g., lean body mass) to support the utilization of calories for weight loss and healthy weight maintenance. Whey proteins are an excellent source of branched chain amino acids which support tissue repair as well as synthesis of hormones, enzymes, and other compounds critical to biochemical processes in the body. Additionally, protein provides bioactive compounds including immunoglobulins and peptides that support gut health for immune system benefits and improved nutrient absorption.

Whey protein concentrate is preferably provided in an amount ranging from 12.66871 wt. % to 50.67482 wt. % of the composition. In certain embodiments, whey protein concentrate is provided in an amount ranging from 13.935581 wt. % to 45.607338 wt. %. In additional embodiments, whey protein concentrate is provided in an amount ranging from 15.202452 wt. % to 40.539856 wt. %. In still further embodiments, whey protein concentrate is provided in an amount ranging from 16.469323 wt. % to 35.472374 wt. %.

Whey protein isolate is preferably provided in an amount ranging from 12.5407 wt. % to 50.16294 wt. % of the composition. In certain embodiments, whey protein isolate is provided in an amount ranging from 13.794770 wt. % to 45.146646 wt. %. In additional embodiments, whey protein isolate is provided in an amount ranging from 15.048840 wt. % to 40.130352 wt. %. In further embodiments, whey protein isolate is provided in an amount ranging from 16.302910 wt. % to 35.114058 wt. %.

### Leucine

GLP-1 appetite suppression with reduced protein intake lowers dietary leucine availability. However, an intake of an adequate amount of leucine is essential to initiate protein synthesis for muscle preservation and supporting a healthy metabolic rate that is vital to achieving and sustaining a healthy weight. Leucine helps encourage fitness and physical activity, as it reduces activity-related muscle soreness that those who exercise intensely or those who are newer to exercise are likely to experience. Leucine with vitamin D-enriched formulas, alone or with physical activity, show a positive effect on markers of inflammation and a muscle-protein-sparing effect which is vital for those at risk for losing lean muscle mass (i.e., lean body mass). L-leucine, in particular, is advantageous because it is easily absorbed in the small intestine. In certain preferred embodiments, the supplement includes instantized L-leucine to increase total leucine content to an amount up to approximately 2.52 wt. % of the supplement composition to promote lean body mass during weight loss. In other embodiments, powdered leucine is utilized, although it is not as soluble and therefore not as functional in beverage form. In most preferred embodiments, leucine is used in an amount ranging from an amount greater than 0 wt. % to 2.28512 wt. %. In certain embodiments, leucine is used in an amount ranging from an amount greater than 0 wt. % to 2.056608 wt. %. In additional embodiments, leucine is used in an amount ranging from an amount greater than 0 wt. % to 1.828096 wt. %. In other embodiments, leucine is used in an amount ranging from an amount greater than 0 wt. % to 1.599584 wt. %.

### Ground Flaxseed and Other ALA Sources

The supplement additionally includes healthy fats and oils to supplement those not being consumed due to loss of appetite. The body requires dietary Omega-3 for proper function of the heart, brain, and immune system. Reduced food intake with appetite suppressant drugs could in turn reduce intake of omega-3 fatty acids which are vital for cardiovascular and cognitive health. Therefore, healthy fats and oils should be supplemented if a user of a GLP-1 agonist drug is not consuming the proper amount due to a reduced appetite. Ground flaxseed is an excellent source of Alpha Linoleic Acid (ALA), an essential Omega-3 fatty acid which helps mitigate chronic inflammation and promote health. ALA is readily digested and absorbed by the body. From a practical standpoint, certain forms of ground flaxseed for ALA also provide advantages because they are shelf stable and have sustained functional benefits at the end of a twelve month shelf life. The supplement includes a shelf stable source of ALA including but not limited to dried ahi flower oil, canola oil, pumpkin seed oil, flaxseed oil, ground flaxseed, or soybean oil. In preferred embodiments, however, a specialized ground flaxseed is used, as it has a high concentration of ALA and is more shelf stable than other sources of ALA.

In preferred embodiments, ground flaxseed is provided in an amount ranging from an amount greater than 0 wt. % to 11.425600 wt. %. In certain embodiments, ground flaxseed is provided in an amount ranging from an amount greater than 0 wt. % to 10.283040 wt. %. In other embodiments, ground flaxseed is provided in an amount ranging from an amount greater than 0 wt. % to 9.140480 wt. %. In additional embodiments, ground flaxseed is provided in an amount ranging from an amount greater than 0 wt. % to 7.997920 wt. %.

### Vitamins and Minerals

As those taking GLP-1 agonist medications experience delayed gastric emptying and reduced nutrition intake, vitamin and mineral supplementation is necessary to make up for resulting deficits in the same. For example, reduced iron can lead to greater fatigue, anemia, and lower immunity. Vitamin B12 deficiency often leads to fatigue, weakness, and issues of constipation or digestive gas. However, zinc supplementation has been shown to alleviate diarrhea and enhance gut barrier function through nutrient absorption. Moreover, highly bioavailable sources of zinc include beneficial anti-inflammatory properties that provide protection to cells from oxidative damage and promote immune health.

Vitamin D and vitamin K support calcium utilization. These micronutrients, along with dietary protein, are responsible for maintaining bone density. Low vitamin D status has been associated with greater risk for cardiovascular disease, type 2 diabetes, and metabolic syndrome. In certain embodiments, vitamin D is supplied as vitamin D2 (ergocalciferol), which is derived from any one of plant sources, marine organisms, fungal sources, and yeast sources. In preferred embodiments, vitamin D is provided in the form of vitamin D3 (cholecalciferol), which is obtained from animal sources including fatty fish, liver, egg yolks, and lanolin (sheep's wool). Only vitamin D3 is considered bio-identical to the vitamin D produced in the body through exposure to sunlight. A highly bioavailable source of vitamin D3 is utilized to meet needs for the metabolic functions in the body, including the support of mineral absorption, muscle function, bone health, and immunity. One hundred percent of the 20 microgram recommended daily value is included with each serving of the preferred embodiment of the composition, as vitamin D is often sequestered in fat tissue (adipose), resulting in reduced bioavailability and greater risk of deficiency for overweight individuals. Vitamin D3 is the preferred form of vitamin D for this reason, as it is more efficiently absorbed and utilized in the body than other forms of vitamin D. In the most preferred embodiments, a spray dried natural vitamin D3, such as cholecalciferol, is used that is protected by antioxidants including but not limited to alpha-tocopherol and sodium ascorbate in a form that is fully dispersible in a reconstituted nutritionally formulated product with certified documentation of purity and potency.

Enhanced levels of vitamins A, B12, and magnesium are needed to support the gut lining and production of gastric enzymes to break down food and allow for the absorption of nutrients. In certain embodiments, the vitamin B12 source is stable in a dry powdered blend, has high bioavailability, and is easily digested. Adequate vitamin B12 is needed to address fatigue, constipation, gas, and loss of appetite. Alternative sources of vitamin B12 include cyanocobalamin, hydroxocobalamin, methylcobalamin, and adenosylcobalamin, as well as nutritional yeast, algae, seaweed, and mushroom sources. Moreover, these sources of vitamin B12 may be encapsulated, complexes, or processed by trituration with a variety of excipients. In preferred embodiments, vitamin B12 is provided via a uniform, water soluble powder that meets United States Pharmacopeia (USP) standards of purity. The most preferred form is a highly stable cyanocobalamin of pharmaceutical grade produced by trituration of cyanocobalamin with mannitol as the excipient to achieve a highly consistent product with optimal bioavailability.

In certain embodiments, vitamin A is included in the composition to support vision, cellular differentiation, immune function, and multiple antioxidant functions. Exemplary forms of vitamin A used in the composition include retinyl palmitate (retinoids) and beta-carotene (carotenoid), which could also be derived from various fruits, vegetables, and other foods. In preferred embodiments, vitamin A palmitate is used in a microencapsulated retinol form. The encapsulated form protects vitamin A from oxidation during storage and is also cold water dispersible to support extended release during digestion.

For enhanced nutritional intake, vitamin C and K2 are also optimized in certain embodiments, as vitamin C provides antioxidant and immune support and vitamin K2 utilizes calcium in bone formation and promotes cardiovascular health. In certain embodiments, the source of vitamin C is stable in the dry powdered form, has high bioavailability, and has sustained release characteristics to ensure optimal absorption and utilization in the body to support metabolic and antioxidant functions. Vitamin C fortification in a dry powdered form is provided by methods including but not limited to the addition of ascorbic acid, sodium ascorbate, citrus powders, various fruit powders, and rose hips. In preferred embodiments, the vitamin C source is highly absorbable within the body and standardized to provide a consistent amount per dose. In preferred embodiments, the vitamin C form is a free flowing, fully encapsulated form of USP grade ascorbic acid to protect the vitamin C from interaction with other ingredients in the dry powdered blend. This allows for increased stability and shelf life, and the sustained release form aids in the vitamin C being completely digested within the body. In certain embodiments of the invention, the supplement incorporates a source of bioactive vitamin K2 in combination with vitamin D3 and milk mineral sources of calcium, phosphorous, and magnesium. This combination supports the biochemical process of bone mineralization and inhibits precipitation of calcium in smooth muscle to preserve healthy arterial flexibility and blood transport for cardiovascular function, as well as optimized bone mass and bone strength.

In certain embodiments, vitamin K2 (menaquinone) sources used in the supplement include plant and animal sources including but not limited to soy, algae, liver, egg yolks, meat products, and dairy products including fermented cheese, and fermented soy (natto). In other embodiments, vitamin K2 is used in the supplement that is obtained via bacterial fermentation to yield menaquinones of various chain lengths including MK-4, MK-7, MK-8, and MK-9 with differing chemical structures that alter bioavailability and biological activity in the body. In preferred embodiments, MK4 and MK7 are used as vitamin K2 sources, with the most preferable embodiment including an all trans MK7 derived by natural fermentation to provide the highest quality, purity, and reliability of a readily absorbed form with no risk of allergen or objectionable flavors.

In certain embodiments, vitamin K1 (phytonadione) is utilized in the composition to meet needs for metabolic functions in the body including blood clotting, bone metabolism, and cellular regulation. Vitamin K1 may take the form of phylloquinone (natural Vitamin K) and phytonadione, a manufactured version. Phytonadione provides standardization for formulating products, as natural sources may have more varied levels when derived from leafy greens, vegetables, or herbs. Thus, preferred embodiments include phytonadione to enhance stability and uniform distribution.

In certain embodiments, choline is provided within the supplement. Choline is an essential nutrient in physiological functions including neurotransmission, lipid metabolism, and promotion of healthy cell structure. While present in a variety of foods, choline processing steps for the form of choline used requires optimized bioavailability to assure ease of absorption for effective utilization in the body to meet dietary needs. In certain embodiments, dietary sources of choline include egg yolk, liver, fish, seafood soy lecithin, and other sources of lecithin. One or more of these dietary sources can be utilized to produce a standardized form of choline by extraction with L-tartaric acid in a controlled reaction to have minimum flavor and optimal digestibility. In preferred embodiments, choline is provided in a uniform white crystalline power that meets USP standards of purity. The most preferred form of choline is a fine mesh powder of choline bitartrate from natural L-tartaric acid with a purity of at least 98% after passing through a 40 mesh screen to assure even distribution in the composition and high bioavailability within the body.

GLP-1 receptor agonist medications cause delayed gastric emptying and inhibit duodenal and small intestine motility. As a result, absorption of fat soluble vitamins A, D, E, and K will be negatively impacted. The disclosed supplement, therefore, is to be taken alongside GLP-1 agonist medications and aims to address the negative GLP-1 agonist digestive side effects of lower micronutrient absorption by incorporating soluble prebiotic fibers to promote digestive and gut health to address micronutrient deficiencies related to GLP-1 agonist medications.

In certain embodiments, the vitamins in the supplement are unencapsulated. In other embodiments, encapsulated vitamins are used in the supplement to optimize protection and stability of the vitamins that will be easily digested. Therefore, this provides for high bioavailability of said vitamins and their easy absorption during transit through the digestive tract to provide for a full range of B vitamins as well as vitamins A, C, D, E, and K. Encapsulation of vitamins in the supplement is carried out via methods including but not limited to spray drying, fluid bed coating, and utilization of lipids, alginates, or other plant-derived material to create protective bi-layers, reducing said vitamins from exposure to environmental degradative factors in the air or from other food ingredients in the supplement. In preferred embodiments, the vitamins will be stable for a minimum of 12 months, most preferably 24 months or more, with minimal impact on the organoleptic profile of the product, protecting the active ingredients until consumed. Together, the aforementioned factors promote bioavailability to meet the essential vitamin requirements in the body. The preferred amounts of these vitamins are at minimum 50% daily value (DV) with vitamins C, D, and B12 at or above 100% DV in order to combat deficiency symptoms and lead to better overall weight loss results.

In preferred embodiments, vitamin C is provided in an amount ranging from 0.635130 wt. % to 1.27026 wt. %. In certain embodiments, vitamin C is provided in an amount ranging from 0.698643 wt. % to 1.143234 wt. %. In other embodiments, vitamin C is provided in an amount ranging from 0.762156 wt. % to 1.016208 wt. %. In still further embodiments, vitamin C is provided in an amount ranging from 0.825669 wt. % to 0.889182 wt. %.

In certain embodiments, vitamin E is included in the composition to promote antioxidant protection, immune support, and anti-inflammatory functions to protect cell tissue. Examples of forms of vitamin E used in the composition include at least one of alpha-tocopherol, gamma-tocopherol, delta-tocopherol and/or tocotrienols. The most preferred form is an all-rac-alpha tocopheryl acetate vitamin E dispersed in an encapsulation matrix which protects stability during storage. This form of vitamin E is a cold-water dispersible powder to allow for an expedient absorption rate during digestion. In preferred embodiments, vitamin E is provided in an amount ranging from 0.021705 wt. % to 0.086820 wt. %. In certain embodiments, vitamin E is provided in an amount ranging from 0.023876 wt. % to 0.078138 wt. %. In other embodiments, vitamin E is provided in an amount ranging from 0.026046 wt. % to 0.069456 wt. %. In additional embodiments, vitamin E is provided in an amount ranging from 0.028217 wt. % to 0.060774 wt. %.

In preferred embodiments, vitamin B6 is provided in an amount ranging from 0.001355 wt. % to 0.005420 wt. %. In other embodiments, vitamin B6 is provided in amounts ranging from 0.001491 wt. % to 0.004878 wt. %; 0.001626 wt. % to 0.004336 wt. %; or 0.001762 wt. % to 0.003794 wt. %. Vitamin B12 is provided in an amount preferably ranging from 0.000005 wt. % to 0.000020 wt. %. In other embodiments, vitamin B12 is provided in an amount ranging from 0.000006 wt. % to 0.000018 wt. %; 0.000006 wt. % to 0.000016 wt. %; and 0.000007 wt. % to 0.000014 wt. %.

In preferred embodiments, vitamin A is provided in an amount ranging from 0.001270 wt. % to 0.005080 wt. %. In certain embodiments, vitamin A is provided in an amount ranging from 0.001397 wt. % to 0.004572 wt. %. In other embodiments, vitamin A is provided in an amount ranging from 0.001524 wt. % to 0.004064 wt. %. In further embodiments, vitamin A is provided in an amount ranging from 0.001651 wt. % to 0.003556 wt. %.

In still further preferred embodiments, vitamin K is provided in an amount ranging from 0.000080 wt. % to 0.000320 wt. %. While other forms of vitamin K may be used, preferred embodiments provide vitamin K2 in an amount ranging from 0.000015 wt. % to 0.000060 wt. %. In certain embodiments, vitamin K2 is provided in an amount ranging from 0.000017 wt. % to 0.000054 wt. %; 0.000018 wt. % to 0.000048 wt. %; or 0.000020 wt. % to 0.000042 wt. %.

Finally, in preferred embodiments, vitamin D is provided in an amount ranging from 0.000025 wt. % to 0.000100 wt. %. In certain embodiments, vitamin D is provided in an amount ranging from 0.000028 wt. % to 0.000090 wt. %. In other embodiments, vitamin D is provided in an amount ranging from 0.000030 wt. % to 0.000080 wt. %, or more specifically 0.000033 wt. % to 0.000070 wt. %.

The supplement also includes minerals such as mineral chelates, complexes, and salts. In certain embodiments, the supplement includes a customized blend of minerals, including trace minerals, to support bodily needs for calcium, iron, potassium, phosphorus, magnesium, sodium, copper, zinc, selenium, manganese, chromium, molybdenum, and iodine in the supplement's macronutrient components. Daily mineral intakes are of concern for overweight individuals who may be nutritionally compromised due to poor dietary intake. While pills or whole foods can provide minerals to the body, these sources may also contain materials that reduce or limit mineral absorption such as phytates, oxalates, and caffeine. As a result, preferred mineral sources of the supplement include high purity mineral sources with documentation to be free of toxic or harmful substances, with the most preferred embodiments including minerals with highest bioavailability for absorption and utilization in the body, such as chelated forms.

In certain embodiments, the composition includes forms of calcium including calcium carbonate, citrate, phosphate, gluconate or lactate. In other examples, a milk calcium complex is primarily a source of calcium but also provides phosphorous, potassium, and magnesium along with the trace minerals zinc, copper, and iron that are easily and readily digestible and stable in solution. The preferred source minerals will have one source contribute several minerals and are shelf stable, easily digested, and stable in a beverage solution. For example, phosphorous is included in the milk mineral complex in exemplary embodiments in forms including but not limited to monopotassium phosphate, tricalcium phosphate, disodium phosphate, calcium phosphate, and a variety food sources with naturally occurring phosphorus including dairy products and animal and plant based food. The most preferred milk mineral complex is a natural dairy source which has been validated by clinical studies, such as TRUCAL. TRUCAL includes a proportion of calcium, phosphorus, magnesium, potassium, and trace minerals zinc, copper, and iron which are optimally balanced for bone support.

In preferred embodiments, calcium is provided in the form of a milk calcium complex suitable for beverage use in an amount ranging from 2.856400 wt. % to 5.712800 wt. %. In certain embodiments, calcium is provided in an amount ranging from 3.142040 wt. % to 5.141520 wt. %. In additional embodiments, calcium is provided in an amount ranging from 3.427680 wt. % to 4.570240 wt. %. In further embodiments, calcium is provided in an amount ranging from 3.713320 wt. % to 3.998960 wt. %.

In certain embodiments, potassium is provided in the composition. Preferably, the potassium source is easily bioavailable and digestible and is palatable to the user when consumed. In certain embodiments, potassium is provided in the form of potassium chloride, potassium citrate, potassium bicarbonate, potassium phosphate, and/or a variety of food sources with naturally high potassium content including dairy products such as milk minerals, fruits, vegetables, and whole grains. In preferred embodiments, dipotassium phosphate or milk minerals are used in the composition, as both forms have high stability and buffering properties to help stabilize the reconstituted product. In the most preferred embodiments, potassium is provided in the form of dipotassium phosphate in an amount ranging from 1.69099 wt. % to 6.76396 wt. %. In certain embodiments, potassium is provided in an amount ranging from 1.860089 wt. % to 6.087564 wt. %. In other embodiments potassium is provided in an amount ranging from 2.029188 wt. % to 5.411168 wt. %. In additional embodiments, potassium is provided in an amount ranging from 2.198287 wt. % to 4.734772 wt. %.

In certain embodiments, magnesium is provided in the composition. In order to minimize gastrointestinal discomfort, the magnesium source is preferably easily dissolvable and capable of staying suspended in solution without negatively impacting the taste of the composition. In certain embodiments, magnesium is provided in the form of at least one of magnesium oxide, magnesium glycinate, magnesium lactate, magnesium citrate, magnesium acetyltaurate, magnesium sulfate, magnesium carbonate and magnesium gluconate. In preferred embodiments, magnesium is provided in the form of magnesium bisglycinate dihydrate in an amount ranging from 0.856920 wt. % to 3.427680 wt. % and magnesium lactate in an amount ranging from 0.856920 wt. % to 3.42768 wt. %. Magnesium lactate is used not only for its physical qualities that make incorporation into beverage forms easier, but also for its higher digestive tolerability. However, magnesium bisglycinate dihydrate is most preferred because the amino acid chelate form is most easily transported across the intestinal wall while having the greatest success at minimizing digestive upset. In certain embodiments, magnesium bisglycinate dihydrate is provided in an amount ranging from 0.942612 wt. % to 3.084912 wt. %; 1.028304 wt. % to 2.742144 wt. %; or 1.113996 wt. % to 2.399376 wt. %. In certain embodiments, magnesium lactate is provided in an amount within the ranges of 0.942612 wt. % to 3.084912 wt. %; 1.028304 wt. % to 2.742144 wt. %; or 1.113996 wt. % to 2.399376 wt. %.

In certain embodiments, iron is provided in the composition. The iron source is preferably highly palatable, bioavailable, and digestible. In preferred embodiments, the iron source is encapsulated to prevent negative interaction with other ingredients and avoid gastric distress. In certain embodiments, iron is provided in the form of ferrous sulfate, fumarate, gluconate, iron bisglycinate, iron polysaccharide, or carbonyl iron. Preferred iron sources are uniform, water soluble powders that meet USP standards of purity. In preferred embodiments, iron is provided in the form of fine mesh encapsulated ferrous sulfate in an amount ranging from 0.069660 wt. % to 0.278640 wt. %. In most preferred embodiments, the ferrous sulfate is of at least 98% purity, which is achieved by passing the ferrous sulfate through a 30 mesh screen to achieve optimal distribution in the composition and extended release bioavailability. In certain embodiments, iron is provided in an amount ranging from 0.076626 wt. % to 0.250776 wt. %. In other embodiments, iron is provided in an amount ranging from 0.083592 wt. % to 0.222912 wt. %. In further embodiments, iron is provided in an amount ranging from 0.090558 wt. % to 0.195048 wt. %.

In certain embodiments, copper is provided in the composition. Again, preferred forms of copper minimize digestive discomfort while being easily digestible (e.g., capable of being absorbed at a low dose amount) and capable of staying suspended in solution. Sources of copper used in certain embodiments include at least one of copper glycinate, copper gluconate, copper carbonate, copper oxide, and/or a variety of food sources that are naturally high in copper including liver, spirulina (algae), shiitake mushrooms, seafood, nuts, seeds, leafy greens, and whole grains. The most preferred form is microencapsulated copper gluconate, a highly stable form of copper that is encapsulated to mask the taste and odor of the copper, prevent degradation, and minimize interaction with other ingredients while supporting iron absorption, formation of red blood cells, and proper bone formation and maintenance. In preferred embodiments, copper is provided in the form of copper gluconate in an amount ranging from 0.004320 wt. % to 0.017280 wt. %. In certain embodiments, copper is provided in an amount ranging from 0.004752 wt. % to 0.015552 wt. %. In other embodiments, copper is provided in an amount ranging from 0.005184 wt. % to 0.013824 wt. %. In additional embodiments, copper is provided in an amount ranging from 0.005616 wt. % to 0.012096.

In certain embodiments, manganese is provided in the composition. Again, preferred forms of manganese minimize digestive discomfort while being easily digestible (e.g., capable of being absorbed at a low dose amount) and capable of staying suspended in solution. Examples of forms of manganese include manganese gluconate, manganese sulfate, manganese citrate, and/or a variety of food sources including but not limited to nuts, seeds, whole grains, legumes, seafood, leafy greens, fruits, and spices. In preferred embodiments, manganese sulfate is used for its water solubility, allowing it to be easily incorporated into a reconstituted beverage. This form is easily absorbable at low doses with excellent stability, assuring nutrient content will be maintained over the composition's shelf-life. In certain embodiments, manganese is provided in an amount ranging from 0.004330 wt. % to 0.017320 wt. %, with preferred embodiments including an amount of 0.008660 wt. %. In certain embodiments, manganese is provided in an amount within the ranges of 0.004763 wt. % to 0.015588 wt. %; 0.005196 wt. % to 0.013856 wt. %; and 0.005629 wt. % to 0.012124 wt. %.

In certain embodiments, chromium is provided in the composition. Preferred chromium sources are odorless, tasteless, and heat-stable to support proper insulin function, control blood sugar, and promote weight control and lean body mass. In certain embodiments, chromium is provided in the form of at least one of chromium polynicotinate, chromium chloride, chromium citrate, chromium picolinate, and a variety of food sources including but not limited to nutritional yeast and some nuts, whole grains, and vegetables. Preferred embodiments use chromium polynicotinate, as it is a potent niacin-bound chromium complex. Examples of commercial products providing chromium polynicotinate include CHOMEMATE. In certain embodiments, chromium is provided in an amount ranging from 0.000210 wt. % to 0.000840 wt. %, with preferred embodiments including chromium in an amount of 0.000420 wt. %. In certain embodiments, chromium is also provided in an amount within the ranges of 0.000231 wt. % to 0.000756 wt. %; 0.000252 wt. % to 0.000672 wt. %; and 0.000273 wt. % to 0.000588 wt. %.

In certain embodiments, selenium is provided in the composition. Some examples of selenium forms include inorganic forms (sodium selenite and selenate) and organic forms (selenium methylselenocysteine and selenium-enriched yeast). Other examples of food sources that contain nutritional selenium include but are not limited to nuts such as Brazil nuts, fish, seafood, and egg yolk depending on water and/or food sources. In preferred embodiments, L-Selenomethionine is used, which is a selenium-integrated chelated form of methionine in the L(+) form and the only isomer of amino acids used to synthesize proteins. This allows L-Selenomethionine to substitute for methionine in protein synthesis, making it more metabolically active than other forms. In certain embodiments, selenium is provided in an amount ranging from 0.000080 wt. % to 0.000320 wt. %, with preferred embodiments including an amount of 0.000160 wt. %. Certain embodiments include selenium provided in an amount within the ranges of 0.000088 wt. % to 0.000288 wt. %; 0.000096 wt. % to 0.000256 wt. %; and 0.000104 wt. % to 0.000224 wt. %.

In certain embodiments, molybdenum is provided in the composition. Examples of forms of molybdenum include molybdenum glycinate, molybdenum chloride, and molybdenum picolinate. The most preferred form is molybdenum glycinate chelate, as it is bound to the amino acid glycine and therefore is more easily absorbed within the body. In certain embodiments, molybdenum is provided in an amount within the range of 0.000190 wt. % to 0.000760 wt. %, with preferred embodiments having an amount of 0.00038 wt. %. In certain embodiments, molybdenum is provided in an amount within the ranges of 0.000209 wt. % to 0.000684 wt. %; 0.000228 wt. % to 0.000608 wt. %; and 0.000247 wt. % to 0.000532 wt. %.

In certain embodiments, iodine is provided in the composition in form that is easily controlled at very low levels of intake to prevent deficiency. In certain embodiments, iodine is provided in the form of potassium iodide and sodium iodide as added to iodized salt, seafood, seaweed, algae, dairy products, or eggs depending on the iodide content of animal feed. Preferred embodiments use potassium iodine for its safety, solubility, and ability to not loose potency over time. In certain embodiments, iodine is provided in an amount ranging from 0.000135 wt. % to 0.000540 wt. %, with preferred embodiments including an amount of 0.000270 wt. %. In certain embodiments, iodine is provided in an amount within the ranges of 0.000149 wt. % to 0.000486 wt. %; 0.000162 wt. % to 0.000432 wt. %; and 0.000176 wt. % to 0.000378 wt. %.

In certain embodiments, zinc is provided in the composition. Examples of the forms of zinc suitable for the composition include zinc oxide, zinc sulfate, zinc carnosine, and zinc picolinate. Use of zinc carnosine is preferred at levels effective for addressing gastrointestinal side effects and, in combination with zinc sulfate monohydrate, providing the total elemental zinc content present in the product. For example, zinc carnosine is used in certain embodiments as an anti-emetic agent. In preferred embodiments, zinc is provided in the form of zinc carnosine—separately or with zinc sulfate—in an amount greater than 0.001275 wt. % up to 0.0051 wt. %. In certain embodiments, zinc is provided in an amount ranging from 0.001403 wt. % to 0.004590. In additional embodiments, zinc is provided in an amount ranging from 0.001530 wt. % to 0.004080 wt. %. In other embodiments, zinc is provided in an amount ranging from 0.001658 wt. % to 0.003570.

### Digestively Stable Probiotics and Digestive Botanical Extracts

Digestively stable probiotics are associated with enhancing nutrient absorption while reducing bloating and gastric discomfort. These probiotics rebalance bacteria in the gut microbiome for improved digestive health and well-being. Probiotics aid in calming an upset stomach and manage indigestion symptoms including but not limited to nausea, feelings of fullness, including upper abdominal fullness, gas, bloating, and heartburn. Thus, the disclosed supplement works synergistically with probiotics to lower these gastrointestinal symptoms to maintain normal digestion and healthy bowel movements, thereby reliving constipation. In preferred embodiments, probiotics function as an anti-bloating agent, anti-diarrheal agent, and anti-constipation agent.

In certain inventive embodiments, the supplement includes probiotic strains with digestive resilience to remain viable under adverse, low pH conditions in the stomach to reach and colonize in the intestinal tract. These strains provide benefits including the creation of short chain fatty acids which benefit cells in the intestinal lining and support an advantageous balance of intestinal organisms, which enhance nutrient absorption while promoting digestive comfort, immunity, and regularity with reduced incidences of bloating, diarrhea, and constipation. In additional embodiments, probiotic strains such as bacillus, lactobacillus, Bifidobacterium, and similar bacteria and yeast are used which aid in providing probiotic benefits to digestive health. Inclusion of a resistant Bacillus is preferred for its stability and resilience to changes in moisture and temperature, with the most preferred embodiments of the supplement including B. coagulans MTCC5856, used at 500,000,000 CFU in each of two servings per day to provide 1 billion colony forming units (CFU) per day. Through use of probiotics, the supplement reduces bloating and gastric discomfort by enhancing nutrient absorption and supporting a beneficial balance of health promoting bacteria in the gut microbiome during the period of a user consuming the supplement.

In certain embodiments of the supplement, the composition includes Glycyrrhiza glabra (licorice root), caraway, and L-menthol extracts. However, in preferred embodiments, the composition includes neutral-tasting bioactive Glycyrrhiza glabra extract in an amount ranging from an amount greater than 0 wt. % to 0.171380 wt. %, which has been clinically shown to safely and effectively manage symptoms of functional dyspepsia including upper abdominal fullness and pain, nausea, bloating, and vomiting. In certain embodiments, Glycyrrhiza glabra extract is used in the supplement as at least one of an anti-emetic agent, anti-constipation agent, and anti-bloating agent. In certain embodiments, Glycyrrhiza glabra extract is provided in an amount ranging from an amount greater than 0 wt. % to 0.154242 wt. %. In other embodiments, Glycyrrhiza glabra extract is provided in an amount ranging from an amount greater than 0 wt. % to 0.137104 wt. %. In further embodiments, Glycyrrhiza glabra extract is provided in an amount ranging from an amount greater than 0 wt. % to 0.119966 wt. %. In most preferred embodiments, the use of select Glycyrrhiza glabra extract is preferred for its compatibility with probiotics and digestive enzymes without impacting their activity levels. For example, GUTGARD is a clinically researched natural bioactive for daily digestive health.

### Digestive Enzymes

Digestive enzymes include enzymes derived from animal, plant, fungal, or microbial sources including but not limited to α-amylase, protease, lipase, cellulase, peptidase, xylanase, galactosidase, and lactase, which promote proper digestion, enhance nutrient absorption, and relieve gastrointestinal symptoms. These enzymes facilitate beneficial access to dietary carbohydrates, proteins, fats, plant fibers, and lactose to meet nutritional needs. As examples, protease will support pre-digestion of the whey protein to facilitate amino acid bioavailability and create smaller peptides to help prevent gas, diarrhea, and bloating. The natural properties of the stomach include gastroprotective mechanisms to protect the stomach lining from damages due to stress. In preferred embodiments, microbial enzymes are used because they are more stable under a range of environmental conditions (e.g., pH and temperature) and are produced with more consistent enzymatic activity than their plant and animal-derived counterparts. The most preferred embodiments include a multi-enzyme complex consisting mainly of amylase (starch hydrolyzing enzyme), protease (protein hydrolyzing enzyme), lipase (fat hydrolyzing enzyme), cellulase (which hydrolyzes cellulose), and lactase (which hydrolyzes lactose). An example of a multi-enzyme complex includes DIGEZYME. In certain embodiments, the digestive enzymes act as at least one of a anti-emetic agent and anti-bloating agent.

Regarding amylase, certain embodiments use β-amylase and 7-amylase, but α-amylase is used in preferred embodiments. In certain preferred embodiments, aspergillus oryzae-derived α-amylase is utilized in the composition, which hydrolyzes carbohydrates (starches) into dextrin, reducing bloating and gas. Use of α-amylase is preferred because it has a broad pH and temperature activity range; it is also capable of acting at any site along a starch chain, making it faster acting compared to alternative starch-digesting enzymes. In preferred embodiments, amylase is provided is an amount ranging from an amount greater than 0 wt. % to 0.020220 wt. %. In certain embodiments, amylase is provided is an amount ranging from an amount greater than 0 wt. % to 0.018198 wt. %. In other embodiments, amylase is provided is an amount from within the ranges of an amount greater than 0 wt. % to 0.016176 wt. % or an amount greater than 0 wt. % to 0.014154 wt. %.

In certain additional embodiments, protease is used. While alternatives to protease, such as plant-derived enzymes (bromelain derived from pineapple; papain derived from papaya) and proteolytic enzymes (e.g., peptidase and trypsin) are used in certain embodiments, neutral protease is preferred with digestive activity that allows for relative stability in the product after hydration without causing separation due to protein degradation upon mixing with water. The neutral protease is also active in a broad range of conditions and effective in breaking down a wide range of proteins to deliver digestive benefits. In preferred embodiments, the supplement includes protease derived from Bacillus subtilis, with action similar to intestinal proteolytic enzymes which are active in a neutral to alkaline pH range, which helps to break down polypeptides into peptides and amino acids, improving the digestibility and solubility of protein sources, alleviating digestive discomfort, increasing absorption, and promoting gut health. In preferred embodiments, protease is provided in an amount ranging from an amount greater than 0 wt. % to 0.010630 wt. %. In certain embodiments, protease is provided in an amount ranging from an amount greater than 0 wt. % to 0.009567 wt. %. In other embodiments, protease is provided is an amount from within the ranges of an amount greater than 0 wt. % to 0.008504 wt. % or an amount greater than 0 wt. % to 0.007441 wt. %.

In additional embodiments, the supplement includes zinc sources including but not limited to zinc, zinc oxide, and zinc picolinate to promote the production of further digestive enzymes. In certain preferred embodiments, PepZinGI (zinc-carnosine) is included within the composition as a highly bioavailable source of elemental zinc that relieves symptoms of dyspepsia, gastritis, bloating, nausea, and vomiting. In additional preferred embodiments, PepZinGI is used in combination with zinc sulfate monohydrate, providing the total elemental zinc content present in the composition.

### Medium Chain Triglycerides (MCT)

The composition additionally includes medium chain triglycerides (MCT). MCTs support healthy gut linings by supporting the metabolism and encouraging beneficial absorption of dietary fat-soluble vitamins. In certain embodiments, the MCT is a nutritionally available fatty acid with a chain length ranging from 6 to 12 carbon atoms to support metabolic energy. In additional embodiments, the MCT is derived from nutritional oil sources selected from the group including palm, palm kernel, coconut, and dairy fats which contain a combination of caproic (C6), caprylic (C8), capric (C10), and lauric (C12) fatty acids. In preferred embodiments, the MCT includes chain lengths of C8 (caprylic acid) and C10 (capric) fatty acids, with the most preferred being spray dried micro-encapsulates in agglomerated forms utilizing diary protein, such as sodium caseinate to support optimal solubility and stability. In preferred embodiments, MCTs are provided in an amount ranging from an amount greater than 0 wt. % to 1.668190 wt. %. In certain embodiments, MCTs are provided in an amount ranging from an amount greater than 0 wt. % to 1.501371 wt. %. In other embodiments, MCTs are provided in an amount ranging from an amount greater than 0 wt. % to 1.334552 wt. %. In additional embodiments, MCTs are provided in an amount ranging from an amount greater than 0 wt. % to 1.167733 wt. %.

### Dietary Fiber

In certain embodiments, the composition includes soluble fiber. There are many forms and types of fiber that are soluble such as digestion-resistant maltodextrin but come with side effects. Partially hydrolyzed guar gum, for example, does not alter the taste and function of beverages, gels, and solids but can cause gastrointestinal side effects such as gas and diarrhea in some individuals. Inulin is another choice for soluble fiber, but similar to partially hydrolyzed guar, has been shown to cause greater GI distress. Insoluble fibers are also used, but these embodiments are less preferred, as insoluble fibers have little prebiotic benefit. Therefore, preferred embodiments use soluble fibers such as digestion-resistant maltodextrin which provide prebiotic benefits to the gut microbiome including a micronutrient status with lower incidence of gastrointestinal side effects. In preferred embodiments, the dietary fiber used qualifies as a prebiotic. However, it is to be understood that not all dietary fiber qualifies as a prebiotic, as prebiotics are nondigestible food ingredients that support the growth and function of healthy microorganisms that live in the gut, also known as probiotics. Thus, prebiotics support probiotic growth. Additional preferred embodiments use FIBERSOL prebiotic fiber which is a digestion-resistant maltodextrin having a clean taste, excellent solubility, supports post-meal blood glucose, maintains healthy triglyceride levels, promotes regularity, and promotes beneficial bacterial balance in the gut which is associated with an enhanced micronutrient status and immunity. In preferred examples, dietary fiber is provided in the form of digestion-resistant maltodextrin in an amount ranging from 2.513630 wt. % to 20.109060 wt. %. In certain embodiments, dietary fiber is provided in an amount ranging from 2.764993 wt. % to 18.098154 wt. %. In certain embodiments, dietary fiber is provided in an amount ranging from 3.016356 wt. % to 16.087248 wt. %. In additional embodiments, dietary fiber is provided in an amount ranging from 3.267719 wt. % to 14.076342 wt. %. In certain embodiments, dietary fiber is used as an anti-diarrheal agent and anti-constipation agent.

In certain embodiments, the supplement includes the ingredients within the ranges (in wt. %) as shown in Table 1.

TABLE 1
Primary Ingredient Quantities Per Serving

| Ingredient | Active Component | Min (wt. %) | Max (wt. %) |
|---|---|---|---|
| Whey Protein Concentrate | Protein | 12.668710 | 50.674820 |
| Whey Protein Isolate | Protein | 12.540700 | 50.162940 |
| Digestion-resistant Maltodextrin | Dietary Fiber | 2.513630 | 20.109060 |
| Milk Calcium Complex | Calcium | 2.856400 | 5.712800 |
| Dipotassium Phosphate | Potassium | 1.690990 | 6.763960 |
| Flaxseed | ALA | >0.000000 | 11.425600 |
| Magnesium Bisglycinate Dihydrate | Magnesium | 0.856920 | 3.427680 |
| Magnesium Lactate | Magnesium | 0.856920 | 3.427680 |
| Choline Bitartrate | Choline | 0.845495 | 3.381980 |
| Medium Chain Triglycerides | MCT | >0.000000 | 1.668190 |
| Ascorbic Acid | Vitamin C | 0.635130 | 1.270260 |
| Leucine | Leucine | >0.000000 | 2.285120 |
| Glycyrrhiza Glabra Extract | Glycyrrhiza Glabra Extract | >0.000000 | 0.171380 |
| Ferrous Sulfate | Iron | 0.069660 | 0.278640 |
| Zinc Carnosine | Zinc Carnosine | >0.000000 | 0.057130 |
| Alpha Tocopherol Acetate | Vitamin E | 0.021705 | 0.086820 |
| Niacinamide | Niacin | 0.010745 | 0.042980 |
| Amylase | Amylase | >0.000000 | 0.020220 |
| Protease | Protease | >0.000000 | 0.010630 |
| Bacillus coagulans MTCC 5856 | Probiotic | 0.004915 | 0.019660 |
| Manganese Sulfate | Manganese | 0.004330 | 0.017320 |
| Copper Gluconate | Copper | 0.004320 | 0.017280 |
| Lactase | Lactase | >0.000000 | 0.007710 |
| D-Calcium Pantothenate | Pantothenic Acid | 0.003645 | 0.014580 |
| Cellulase | Cellulase | >0.000000 | 0.004950 |
| Pyridoxine Hydrochloride | Vitamin B6 | 0.001355 | 0.005420 |
| Zinc Sulfate | Zinc | >0.001275 | 0.005100 |
| Vitamin A Palmitate | Vitamin A | 0.001270 | 0.005080 |
| Riboflavin | Riboflavin | 0.000900 | 0.003600 |
| Thiamin Mononitrate | Thiamine | 0.000840 | 0.003360 |
| Chromium Polynicotinate | Chromium | 0.000210 | 0.000840 |
| Molybdenum Glycinate Chelate | Molybdenum | 0.000190 | 0.000760 |
| Folic Acid | Folate | 0.000185 | 0.000740 |
| Lipase | Lipase | >0.000000 | 0.000270 |
| Potassium Iodide | Iodine | 0.000135 | 0.000540 |
| L-selenomethionine | Selenium | 0.000080 | 0.000320 |
| Phytonadione | Vitamin K | 0.000080 | 0.000320 |
| Cholecalciferol | Vitamin D | 0.000025 | 0.000100 |
| Biotin | Biotin | 0.000020 | 0.000080 |
| Magnesium Carbonate | Magnesium | 0.000015 | 0.000060 |
| Menaquinone 7 | Vitamin K2 | 0.000015 | 0.000060 |
| Cyanocobalamin | Vitamin B12 | 0.000005 | 0.000020 |

The supplement composition additionally includes secondary ingredients to improve the user experience and allow for easier use in beverage formulations, including sweeteners, gums and stabilizers, and flavorings. In other embodiments, the secondary ingredients allow for easier incorporation into and use within solid formulations or gel formulations or otherwise alter and improve other characteristics of the supplement composition for enhanced consumer acceptance.

In certain embodiments, the supplement composition further includes at least one sweetener selected from natural, processed forms of sugar including but not limited to fructose, honey, agave, botanical extracts of stevia, monk fruit, and other similar plants, and any chemically manufactured high intensity sweeteners including aspartame, sucralose, acesulfame-K, neotame, tagatose, cyclamates, and saccharin. Preferably, the composition includes a customized balance of one or more sweeteners to provide a palatable combination with the protein and bioactive components in the composition while also considering the caloric and glycemic impact of those sweeteners on addressing dietary needs and weight management. In certain exemplary embodiments, the sweeteners are used in a powder to be later added to a liquid to produce a palatable beverage. In other embodiments, composition is in the form of a ready-made beverage wherein the sweeteners are already incorporated and stable in liquid or emulsion form. Likewise, the sweeteners may be used to make a solid or gel formulation more palatable. The preferred natural sweeteners will contribute minimal calories and have low glycemic impact on blood glucose when consumed. The level and balance must be compatible with other ingredients, and sources must have regulatory approval for use, meeting the strict standards of manufacturing and uniformity between lots to assure safety. The most preferred sources of sweeteners include stevia leaf extract and monk fruit extract.

In certain embodiments, the supplement also includes gums and stabilizers. Gums are utilized as thickeners which are selected based on having bland flavor and the ability to provide a desirable, smooth mouthfeel to overcome the drying and particulate nature of ingredients. Stabilizers function to suspend ingredients that will naturally separate out of solution when the supplement is in the form of a liquid or emulsified beverage. Gums and stabilizers that are used include but are not limited to gum acacia, locust bean, pectin, guar gum, carrageenan, konjac, lecithin, starch, gellan, alginates, micro crystalline cellulose, fatty acid esters, or gelatin. In preferred embodiments, the gums and stabilizers will be compatible with other ingredients within the composition and have strict standards of uniformity between lots. The most preferred gums and stabilizers complement the other components in the composition by having bland flavor and achieving the best texture and suspension of ingredients in solution. Embodiments include xanthan, guar gum, and soy lecithin.

While the composition includes a wide variety of natural or artificial flavoring ingredients, certain embodiments use flavorings which enhance the taste and aroma of a chocolate flavored high protein powder which may be later added to a liquid such as water to provide an easily-consumable beverage. Flavorings include cocoa powder, extracts, and other derivatives of cocoa beans and synthetically manufactured nature replicates including extracts or essences that impart chocolate or complementary flavors. In other embodiments, however, alternative flavor profiles including but not limited to vanilla flavoring or fruit flavoring is used. The most preferred flavoring components will be compatible with other ingredients and have strict standards of uniformity between lots.

In preferred embodiments, these secondary ingredients are provided in a total amount ranging from 8.032 wt. % to 32.129 wt. %, with most preferable embodiments having secondary ingredients in a total amount of 16.064 wt. %. Within this total amount, flavor and sweetener secondary ingredients preferably contribute between 7.164 wt. % to 28.655 wt. % of the total secondary ingredients, with most preferred embodiments having flavor and sweetener in an amount of 14.328 wt. %. Moreover, in the same preferred embodiments, textural secondary ingredients preferably contribute to the total secondary ingredients in an amount ranging from 0.868 wt. % to 3.473 wt. %, with most preferred embodiments having textural secondary ingredients in an amount of 1.737 wt. %.

In other embodiments, secondary ingredients are provided in a total amount ranging from 14.458 wt. % to 17.671 wt. %. Of this total amount, flavor and sweetener secondary ingredients contribute amount ranging from 12.895 wt. % to 15.760 wt. %, and textural secondary ingredients contribute an amount ranging from 1.563 wt. % to 1.910 wt. %. In additional embodiments, secondary ingredients are provided in a total amount ranging from 12.852 wt. % to 19.277 wt. %, with flavor and sweetener secondary ingredients contributing to a portion of this total in an amount ranging from 11.462 wt. % to 17.193 wt. % and textural secondary ingredients contributing to a portion of this total in an amount ranging from 1.389 wt. % to 2.084 wt. %. Finally, in still further exemplary embodiments, secondary ingredients are provided in a total amount ranging from 22.949 wt. % to 20.884 wt. %, with flavor and sweetener secondary ingredients contributing to a portion of this total in an amount ranging from 18.626 wt. % to 20.468 wt. % and textural secondary ingredients contributing to a portion of this total in an amount ranging from 2.258 wt. % to 2.481 wt. %.

In certain embodiments, the composition includes tertiary additives incorporated as sub-components of other ingredients. “Tertiary additives” refers to incidental or indirect additives provided in small quantities which serve as functional ingredients or processing aids for other ingredients with no significant nutritional or technical benefit to the finished product. These tertiary additives allow for greater accuracy of and uniformity of the addition of primary and secondary ingredients in the form of carriers, surfactants, and/or free flow agents. In certain embodiments, the tertiary additives include at least one carbohydrate source including but not limited to dextrose; maltodextrin; starch; fat sources including but not limited to lecithin, monoglycerides, and diglycerides; and other miscellaneous compounds including but not limited to microcrystalline cellulose and silicon dioxide. Preferred tertiary additives have no negative impact on taste, texture, or solubility of the finished product. The most preferred tertiary additives will have negligible impact on caloric content and will not introduce any compounds that will contribute to digestive distress. In preferred embodiments, these tertiary additives are provided in an amount ranging from 0.894 wt. % to 3.574 wt. %, with most preferred embodiments including tertiary ingredients in an amount of 1.787 wt. %. In additional exemplary embodiments, tertiary ingredients are provided an amount within the ranges of 1.608 wt. % to 1.966 wt. %; 1.430 wt. % to 2.145 wt. %; and 2.323 wt. % to 2.553 wt. %.

The composition and its elements are manufactured in accordance with current United States Food and Drug Administration (FDA) good manufacturing practices and meet all relevant federal food safety requirements including but not limited to requirements for Chemical Contaminants and Pesticides, Environmental Contaminants, and applicable established Food Defect Action Levels for natural or unavoidable defects in foods for human consumption. [References 1-3].

The invention will be illustrated in more detail with reference to the following Examples, but it should be understood that the present invention is not deemed to be limited thereto.

### EXAMPLES

In one example of the inventive composition, a supplement was prepared by mixing the ingredients in Table 2 (shown in grams of an exemplary 43.76138256 total gram mixture as well as in wt. %) to produce a chocolate flavored powder which is capable of being added to a liquid to produce a beverage.

TABLE 2
Ingredient Quantities per Serving of
Exemplary Powder Supplement

| INGREDIENT | AMOUNT (g) | WT. % |
|---|---|---|
| Primary | 35.94927595 | |
| Nutritive | 35.82581371 | 81.8663% |
| Protein | 22.31400009 | |
| SODIUM CASEINATE* | 0.250000029 | |
| WHEY PROTEIN CONCENTRATE | 11.08800016 | 25.3374% |
| WHEY PROTEIN ISOLATE | 10.9759999 | 25.0815% |
| Amino Acid | 0.500000058 | |
| LEUCINE | 0.500000058 | 1.1426% |
| Fa | 1.730025318 | |
| FLAXSEED | 1.000000115 | 2.2851% |
| MEDIUM CHAIN | 0.730025203 | 1.6682% |
| TRIGLYCERIDES | | |
| Dietary Fiber | 4.400000069 | |
| DIGESTION-RESISTANT | 4.400000069 | 10.0545% |
| MALTODEXTRIN | | |
| Vitamins | 0.588391925 | |
| ALPHA TOCOPHEROL ACETATE | 0.018998129 | 0.0434% |
| ASCORBIC ACID | 0.555882468 | 1.2703% |
| BIOTIN | 1.70669E−05 | 0.0000% |
| CHOLECALCIFEROL | 2.40688E−05 | 0.0001% |
| CYANOCOBALAMIN | 2.62568E−06 | 0.0000% |
| FOLIC ACID | 0.000160167 | 0.0004% |
| MENAQUINONE 7 | 1.26908E−05 | 0.0000% |
| NIACINAMIDE | 0.009406072 | 0.0215% |
| PHYTONADIONE | 6.9143E−05 | 0.0002% |
| PYRIDOXINE HYDROCHLORIDE | 0.001183745 | 0.0027% |
| RIBOFLAVIN | 0.000789893 | 0.0018% |
| THIAMIN MONONITRATE | 0.000736066 | 0.0017% |
| VITAMIN A PALMITATE | 0.001109789 | 0.0025% |
| Minerals | 6.293396258 | |
| CHOLINE BITARTRATE | 0.740000173 | 1.6910% |
| CHROMIUM POLYNICOTINATE | 0.000183798 | 0.0004% |
| COPPER GLUCONATE | 0.003780108 | 0.0086% |
| D-CALCIUM PANTOTHENATE | 0.003188892 | 0.0073% |
| DIPOTASSIUM PHOSPHATE | 1.479999908 | 3.3820% |
| FERROUS SULFATE | 0.060967921 | 0.1393% |
| L-SELENOMETHIONINE | 7.22063E−05 | 0.0002% |
| MAGNESIUM BISGLYCINATE | 0.750000086 | 1.7138% |
| DIHYDRATE | | |
| MAGNESIUM CARBONATE | 1.31284E−05 | 0.0000% |
| MAGNESIUM LACTATE | 0.750000086 | 1.7138% |
| MANGANESE SULFATE | 0.003791049 | 0.0087% |
| MILK CALCIUM COMPLEX | 2.49999985 | 5.7128% |
| MOLYBDENUM GLYCINATE | 0.000165418 | 0.0004% |
| CHELATE | | |
| POTASSIUM IODIDE | 0.000117718 | 0.0003% |
| ZINC SULFATE | 0.001115915 | 0.0026% |
| Digestive Benefits | 0.123462239 | 0.2821% |
| Digestive Enzyme | 0.019162234 | |
| AMYLASE | 0.008849864 | 0.0202% |
| CELLULASE | 0.002167501 | 0.0050% |
| LACTASE | 0.003374878 | 0.0077% |
| LIPASE | 0.000119906 | 0.0003% |
| PROTEASE | 0.004650085 | 0.0106% |
| Herbal Extract | 0.075000009 | |
| LICORICE ROOT EXTRACT | 0.075000009 | 0.1714% |
| Mineral Chelate | 0.025000003 | |
| ZINC CARNOSINE | 0.025000003 | 0.0571% |
| Probiotic | 0.004299993 | |
| BACILLUS COAGULANS MTCC | 0.004299993 | 0.0098% |
| 5856 | | |
| Secondary | 7.029999672 | |
| Non-Nutritive | 7.029999672 | 16.0644% |
| Organoleptic | 6.269999672 | |
| COCOA | 2.49999985 | |
| MONK FRUIT EXTRACT | 0.075000009 | |
| NATURAL FLAVOR | 3.619999804 | |
| STEVIOL GLYCOSIDES (REB M) | 0.075000009 | |
| Technical Function | 0.76 | |
| GUAR GUM | 0.280000207 | |
| XANTHAN GUM | 0.479999793 | |
| Tertiary | 0.782106938 | |
| Indirect Additives | 0.782106938 | 1.7872% |
| Incidental | 0.782106938 | |
| INDIRECT ADDITIVES | 0.782106938 | |
| Grand Total | 43.76138256 | |

*Sodium caseinate functions as carrier on the MCT and therefore is not a primary ingredient, but rather is included in the protein section for accurate substance categorization only.

The exemplary supplement of Table 2, when added to a 6 oz. (177.4 ml) of liquid, produces a low volume beverage with a chocolate flavoring. The exemplary supplement delivers 20 g (40% daily value) of protein with 50% or more of 26 essential micronutrients in one 6 oz. serving, which is a 25% lower volume compared to standard 20 g protein shakes on the market. The lower volume is advantageous for maintaining palatability which is crucial for ensuring maximum patient compliance and ongoing effectiveness.

Moreover, palatability and compliance is encouraged through the use of creating pleasant flavor profiles through natural and artificial flavors as well as achieving a smooth consistency to avoid unpleasant gritty textures often found with other supplements. For example, highly soluble ingredients such as agglomerated proteins, soluble non-viscous fiber sources, and beverage-grade ground flax seed were used to assure uniform distribution of ingredients without separation or settling.

While the invention has been described in detail and with reference to specific embodiments thereof, it will be apparent to one skilled in the art that various changes and modifications can be made therein without departing from the spirit and scope thereof.

### REFERENCES

1. Chemical Contaminants & Pesticides, UNITED STATES FOOD AND DRUG ADMINISTRATION, https://www.fda.gov/food/chemical-contaminants-pesticides (last visited Nov. 20, 2023).
2. Environmental Contaminants in Food, UNITED STATES FOOD AND DRUG ADMINISTRATION, https://www.fda.gov/food/chemical-contaminants-pesticides/environmental-contaminants-food (last visited Nov. 20, 2023).
3. Food Defect Levels Handbook, UNITED STATES FOOD AND DRUG ADMINISTRATION, https://www.fda.gov/food/ingredients-additives-gras-packaging-guidance-documents-regulatory-information/food-defect-levels-handbook #products (last visited Nov. 20, 2023).

1. A composition for consumption by an individual who is being treated with a GLP-1 agonist, said composition comprising:
at least one amino acid source in an amount effective to alleviate loss of lean body mass as a side effect of being treated with the GLP-1 agonist;
at least one dietary fiber; and
a therapeutically effective amount of at least one member selected from the group consisting of a bioactive anti-emetic agent, an anti-bloating agent, an anti-diarrheal agent and an anti-constipation agent,
wherein the composition is a premix or a ready-to-drink supplement effective to alleviate at least one additional side effect of being treated with the GLP-1 agonist, said at least one additional side effect being selected from the group consisting of nausea, bloating, vomiting, diarrhea and constipation.

2. The composition of claim 1, further comprising vitamins, minerals and at least one probiotic.

3. The composition of claim 2, further comprising at least one alpha-linoleic acid (ALA) source, at least one medium chain triglyceride (MCT) having a chain length of 6-12 carbons, leucine, Glycyrrhiza glabra extract, at least one zinc compound, amylase, protease, lactase, cellulase and lipase.

4. The composition of claim 1, comprising:
at least one potassium compound;
at least one magnesium compound;
at least one choline compound;
at least one vitamin;
at least one iron compound;
at least one probiotic;
at least one manganese compound;
at least one copper compound;
at least one chromium compound;
at least one molybdenum compound;
at least one iodine compound; and
at least one selenium compound,
wherein the composition is safe for human consumption.

5. The composition of claim 4, wherein the at least one amino acid source comprises 12-28 grams per 6 oz (177.4 ml) serving of at least one protein, and the at least one dietary fiber comprises at least one prebiotic.

6. The composition of claim 5, wherein:
the at least one amino acid source comprises whey protein concentrate and whey protein isolate;
the at least one calcium compound is milk calcium complex;
the at least one dietary fiber is a soluble fiber that is a digestion-resistant maltodextrin prebiotic;
the at least one potassium compound is dipotassium phosphate;
the at least one magnesium compound comprises magnesium bisglycinate dihydrate and magnesium lactate;
the at least one choline compound is choline bitartrate;
the at least one vitamin comprises ascorbic acid, alpha tocopherol acetate, niacinamide, pyridoxine hydrochloride, vitamin A palmitate, riboflavin, thiamin mononitrate, folic acid, phytonadione, cholecalciferol, menaquinone 7 and cyanocobalamin;
the at least one iron compound is ferrous sulfate;
the at least one probiotic is Bacillus Coagulans MTCC 5856;
the at least one manganese compound is manganese sulfate;
the at least one copper compound is copper gluconate;
the at least one chromium compound is chromium polynicotinate;
the at least one molybdenum compound is molybdenum glycinate chelate;
the at least one iodine compound is potassium iodide; and
the at least one selenium compound is L-Selenomethionine.

7. The composition of claim 6, further comprising at least one alpha-linoleic acid (ALA) source, at least one medium chain triglyceride (MCT) having a chain length of 6-12 carbons, leucine, Glycyrrhiza glabra extract, at least one zinc compound, amylase, protease, lactase, cellulase and lipase.

8. The composition of claim 7, wherein:
the at least one ALA source is flaxseed;
the at least one MCT comprises at least one member selected from the group consisting of caproic acid, caprylic acid, capric acid and lauric acid; and
the at least one zinc compound comprises zinc carnosine and zinc sulfate.

9. The composition of claim 1, wherein each serving comprises:

| Ingredient | Min (g) | Max (g) |
|---|---|---|
| Whey Protein Concentrate | 5.000000 | 20.000000 |
| Whey Protein Isolate | 5.000000 | 20.000000 |
| Digestion-Resistant Maltodextrin | 1.000000 | 8.000000 |
| Flaxseed | >0.000000 | 5.000000 |
| Dipotassium Phosphate | 0.960000 | 2.980000 |
| Milk Calcium Complex | 1.250000 | 2.500000 |
| Magnesium Bisglycinate Dihydrate | 0.375000 | 1.500000 |
| Magnesium Lactate | 0.375000 | 1.500000 |
| Choline Bitartrate | 0.370000 | 1.480000 |
| Leucine | >0.000000 | 1.000000 |
| Medium Chain Triglycerides | >0.000000 | 0.730000 |
| Ascorbic Acid | 0.277941 | 0.555882 |
| Glycyrrhiza Glabra Extract | >0.000000 | 0.075000 |
| Ferrous Sulfate | 0.030484 | 0.060968 |
| Zinc Carnosine | >0.000000 | 0.025000 |
| Alpha Tocopherol Acetate | 0.009499 | 0.018998 |
| Niacinamide | 0.004703 | 0.009406 |
| Amylase | >0.000000 | 0.008850 |
| Bacillus Coagulans MTCC 5856 | 0.002150 | 0.008600 |
| Manganese Sulfate | 0.001896 | 0.007582 |
| Copper Gluconate | 0.001890 | 0.007560 |
| D-Calcium Pantothenate | 0.001595 | 0.006378 |
| Protease | >0.000000 | 0.004650 |
| Lactase | >0.000000 | 0.003375 |
| Pyridoxine Hydrochloride | 0.000592 | 0.002368 |
| Vitamin A Palmitate | 0.000555 | 0.002220 |
| Cellulase | >0.000000 | 0.002168 |
| Riboflavin | 0.000395 | 0.001580 |
| Thiamin Mononitrate | 0.000368 | 0.001472 |
| Lipase | >0.000000 | 0.001200 |
| Zinc Sulfate | >0.000000 | 0.001116 |
| Chromium Polynicotinate | 0.000092 | 0.000368 |
| Molybdenum Glycinate Chelate | 0.000083 | 0.000330 |
| Folic Acid | 0.000080 | 0.000320 |
| Potassium Iodide | 0.000059 | 0.000236 |
| L-Selenomethionine | 0.000036 | 0.000144 |
| Phytonadione | 0.000035 | 0.000138 |
| Cholecalciferol | 0.000012 | 0.000048 |
| Biotin | 0.000009 | 0.000034 |
| Magnesium Carbonate | 0.000065 | 0.000026 |
| Menaquinone 7 | 0.000065 | 0.000026 |
| Cyanocobalamin | 0.000002 | 0.000006 |

10. The composition of claim 9, which is a readily soluble powder premix effective to combine with water to provide a drinkable emulsion.

11. The composition of claim 9, which is a ready-to-drink emulsion further comprising water.

12. The composition of claim 9, which is effective to alleviate loss of lean body mass, nausea, bloating, vomiting, diarrhea and constipation.

13. The composition of claim 1, which is effective to alleviate loss of lean body mass, nausea, bloating, vomiting, diarrhea and constipation.

14. The composition of claim 1, which comprises each of the bioactive anti-emetic agent, the anti-bloating agent, the anti-diarrheal agent and the anti-constipation agent, wherein:
the bioactive anti-emetic agent comprises at least one member selected from the group consisting of at least one digestive enzyme, Glycyrrhiza glabra extract and zinc carnosine;
the anti-bloating agent comprises at least one member selected from the group consisting of Glycyrrhiza glabra extract, at least one probiotic and at least one digestive enzyme;
the anti-diarrheal agent is at least one member selected from the group consisting of at least one dietary fiber and at least one probiotic; and
the anti-constipation agent is at least one member selected from the group consisting of at least one dietary fiber and at least one probiotic.

15. A method for alleviating loss of lean body mass and at least one additional side effect of being administered a GLP-1 agonist, said method comprising feeding the composition of claim 1 to an individual who is being administered the GLP-1 agonist.

16. The method of claim 15, wherein the at least one additional side effect is a member selected from the group consisting of nausea, bloating, vomiting, diarrhea and constipation.

17. The method of claim 15, which alleviates loss of lean body mass, nausea, bloating, vomiting, diarrhea and constipation.

18. The method of claim 15, wherein the composition is fed to the individual in two separate daily servings of a liquid emulsion each of which has a volume of less than 8 ounces (236.6 ml).

19. The method of claim 18, wherein the at least one amino acid source of the composition comprises 12-28 grams per 6 oz (177.4 ml) serving of at least one protein, and the at least one dietary fiber comprises at least one prebiotic.

20. The method of claim 18, wherein each serving comprises:

| Ingredient | Min (g) | Max (g) |
|---|---|---|
| Whey Protein Concentrate | 5.000000 | 20.000000 |
| Whey Protein Isolate | 5.000000 | 20.000000 |
| Digestion-Resistant Maltodextrin | 1.000000 | 8.000000 |
| Flaxseed | >0.000000 | 5.000000 |
| Dipotassium Phosphate | 0.960000 | 2.980000 |
| Milk Calcium Complex | 1.250000 | 2.500000 |
| Magnesium Bisglycinate Dihydrate | 0.375000 | 1.500000 |
| Magnesium Lactate | 0.375000 | 1.500000 |
| Choline Bitartrate | 0.370000 | 1.480000 |
| Leucine | >0.000000 | 1.000000 |
| Medium Chain Triglycerides | >0.000000 | 0.730000 |
| Ascorbic Acid | 0.277941 | 0.555882 |
| Glycyrrhiza Glabra Extract | >0.000000 | 0.075000 |
| Ferrous Sulfate | 0.030484 | 0.060968 |
| Zinc Carnosine | >0.000000 | 0.025000 |
| Alpha Tocopherol Acetate | 0.009499 | 0.018998 |
| Niacinamide | 0.004703 | 0.009406 |
| Amylase | >0.000000 | 0.008850 |
| Bacillus Coagulans MTCC 5856 | 0.002150 | 0.008600 |
| Manganese Sulfate | 0.001896 | 0.007582 |
| Copper Gluconate | 0.001890 | 0.007560 |
| D-Calcium Pantothenate | 0.001595 | 0.006378 |
| Protease | >0.000000 | 0.004650 |
| Lactase | >0.000000 | 0.003375 |
| Pyridoxine Hydrochloride | 0.000592 | 0.002368 |
| Vitamin A Palmitate | 0.000555 | 0.002220 |
| Cellulase | >0.000000 | 0.002168 |
| Riboflavin | 0.000395 | 0.001580 |
| Thiamin Mononitrate | 0.000368 | 0.001472 |
| Lipase | >0.000000 | 0.001200 |
| Zinc Sulfate | >0.000000 | 0.001116 |
| Chromium Polynicotinate | 0.000092 | 0.000368 |
| Molybdenum Glycinate Chelate | 0.000083 | 0.000330 |
| Folic Acid | 0.000080 | 0.000320 |
| Potassium Iodide | 0.000059 | 0.000236 |
| L-Selenomethionine | 0.000036 | 0.000144 |
| Phytonadione | 0.000035 | 0.000138 |
| Cholecalciferol | 0.000012 | 0.000048 |
| Biotin | 0.000009 | 0.000034 |
| Magnesium Carbonate | 0.000065 | 0.000026 |
| Menaquinone 7 | 0.000065 | 0.000026 |
| Cyanocobalamin | 0.000002 | 0.000006 |

21. The method of claim 15, wherein the at least one additional side effect comprises at least two members selected from the group consisting of nausea, bloating, vomiting, diarrhea and constipation.

22. The composition of claim 1, wherein the at least one additional side effect comprises at least two members selected from the group consisting of nausea, bloating, vomiting, diarrhea and constipation.
